(19) United States
(12) Patent Application Publication
(10) Pub. No.: US 2016/0220195 A1
(43) Pub. Date: Aug. 4, 2016

(54) SYSTEM AND METHOD FOR NON-INVASIVE BLOOD PRESSURE MEASUREMENT

(71) Applicant: Midmark Corporation, Versailles, OH (US)

(72) Inventors: Asad Abu-Tarif, Irvine, CA (US); Gongyu Shen, Buena Park, CA (US); Tanvi Pisolkar, Torrance, CA (US); Ruomei Zhang, Cerritos, CA (US); Tony Capparelli, Lake Forest, CA (US)

(21) Appl. No.: 15/005,090

(22) Filed: Jan. 25, 2016

Related U.S. Application Data
(60) Provisional application No. 62/110,704, filed on Feb. 2, 2015.

(51) Int. Cl. A61B 5/00; A61B 5/0225; A61B 5/021; A61B 5/022

(52) U.S. Cl. CPC A61B 5/7278; A61B 5/02233; A61B 5/0225; A61B 5/02116; A61B 5/02125

(57) ABSTRACT

A non-invasive blood pressure (NIBP) monitor is inflated and deflated based upon an algorithm so that a patient's current heart rate may influence the target inflation pressure and the deflation rate. In this manner, if a patient's heart rate is slower than expected, the NIBP monitor may slow its deflation rate so that an appropriate number of cardiac cycles will be captured in order to maximize accuracy. Similarly, if a patient's heart rate is faster than expected, the NIBP monitor may speed its deflation rate to minimize the time of the procedure and the stress on the patient, while still capturing an appropriate number of cardiac cycles.

### PRIORITY

This applications claims priority from U.S. provisional patent application 62/110,704, filed on Feb. 2, 2015 and having the same title as this application. The disclosure of that application is hereby incorporated by reference in its entirety.

### BACKGROUND

In order to acquire an accurate non-invasive blood pressure (NIBP) measurement, a NIBP measurement process must span a sufficient number of cardiac cycles. Some conventional NIBP measurement systems and techniques may operate with a fixed inflation rate and a fixed deflation rate, with both rates remaining the same for each patient. The NIBP measurements may be taken during the deflation phase, which may span a fixed duration in view of the fixed deflation rate. When used with patients having a relatively fast heart rate (HR), a conventional fixed deflation rate may span more cardiac cycles than is needed in order to acquire an accurate NIBP measurement. Such techniques may thus last longer than is needed for patients having a relatively fast HR. When used with patients having a relatively slow HR, a conventional fixed deflation rate may not span enough cardiac cycles to acquire an accurate NIBP measurement. A conventional fixed deflation rate may thus adversely affect speediness or accuracy. It may therefore be desirable to provide a NIBP measurement system that tailors the deflation rate based on the particular patient at hand, thereby improving speediness and/or accuracy of the NIBP measurement.

While a variety of systems and methods have been made and used to obtain NIBP measurements, it is believed that no one prior to the inventors has made or used a system or method as described herein.

### BRIEF DESCRIPTION OF THE DRAWINGS

While the specification concludes with claims which particularly point out and distinctly claim this technology, it is believed this technology will be better understood from the following description of certain examples taken in conjunction with the accompanying drawings, in which like reference numerals identify the same elements and in which:

FIG. 1 depicts a block schematic diagram of a system for non-invasive blood pressure measurement; and

FIG. 2 depicts a flowchart showing various steps of an exemplary method for non-invasive pressure measurement that may be implemented by the system of FIG. 1

The drawings are not intended to be limiting in any way, and it is contemplated that various embodiments of the technology may be carried out in a variety of other ways, including those not necessarily depicted in the drawings. The accompanying drawings incorporated in and forming a part of the specification illustrate several aspects of the present technology, and together with the description serve to explain the principles of the technology; it being understood, however, that this technology is not limited to the precise arrangements shown.

### DETAILED DESCRIPTION

The following description of certain examples of the technology should not be used to limit its scope. Other examples, features, aspects, embodiments, and advantages of the technology will become apparent to those skilled in the art from the following description, which is by way of illustration, one of the best modes contemplated for carrying out the technology. As will be realized, the technology described herein is capable of other different and obvious aspects, all without departing from the technology. Accordingly, the drawings and descriptions should be regarded as illustrative in nature and not restrictive.

It is further understood that any one or more of the teachings, expressions, embodiments, examples, etc. described herein may be combined with any one or more of the other teachings, expressions, embodiments, examples, etc. that are described herein. The following-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

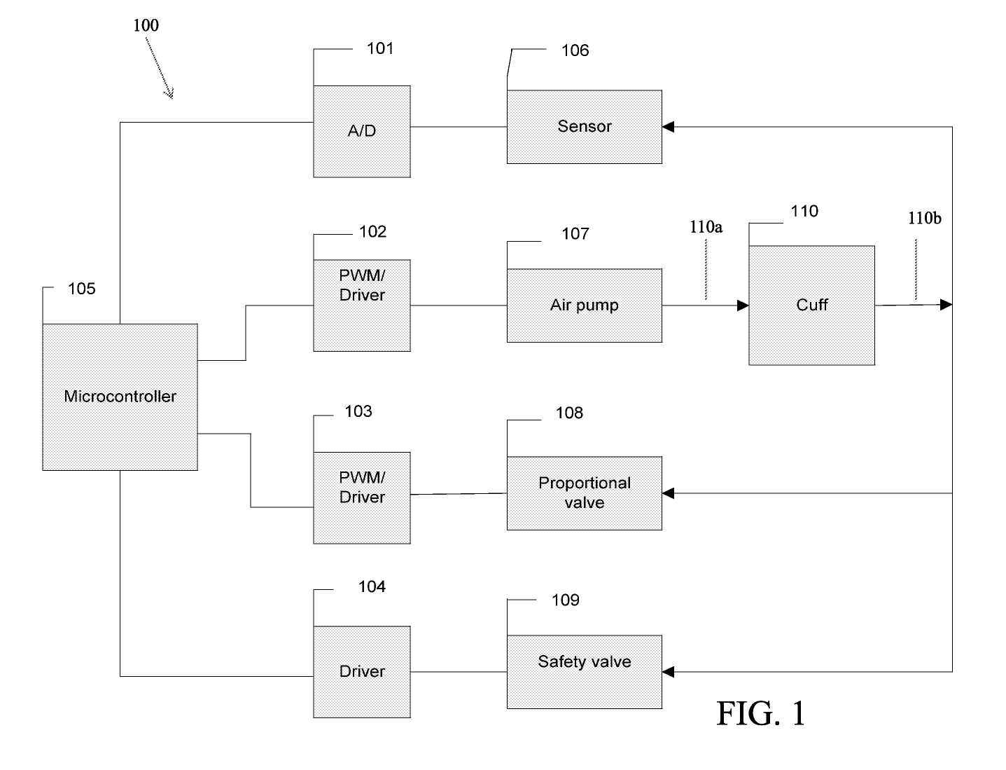

FIG. 1 depicts a block schematic of a system (100) for non-invasive blood pressure (NIBP) measurement according to one example of this disclosure. System (100) of the present example comprises an analog/digital (A/D) converter (101), a pair of pulse width modulation (PWM) drivers (102, 103), a safety valve driver (104), a microcontroller (105), a sensor (106), an air pump (107), a proportional valve (108), a safety valve (109), and a blood pressure cuff (110). As will be described in greater detail below, these components are operable to execute an NIBP measurement algorithm that is tailored on a per patient basis in order to optimize the speed and accuracy of NIBP measurements regardless of whether the patient has a relatively high HR or relatively low HR.

Blood pressure cuff (110) may be configured according to conventional blood pressure cuffs known in the art. For example, cuff (110) may comprise an inflatable cuff that is sized to be fit around a limb of a patient. Moreover, multiple sizes of cuffs (110) may be provided due to the varying limb size of patients, and the importance of using the correct size on a patient that provides the sufficient inflation pressure to obtain an accurate blood pressure reading. As shown, an inlet hose (110a) extends from cuff (110) and provides a path for fluid communication between cuff (110) and air pump (107). Air pump (107) is operable to provide pressurized air to cuff (110) via inlet hose (110a), to thereby inflate cuff (110) as part of a NIBP measurement process. Air pump (107) may comprise a conventional air pump that is typically used in NIBP systems. A one-way valve (e.g., a check valve) may be provided to ensure that air may only flow from air pump (107) toward cuff (110) and not vice-versa. In the present example, air pump (107) is controlled by PWM driver (102). Various suitable forms (i.e., circuit components and configurations) that PWM driver (102) may take will be apparent to those of ordinary skill in the art in view of the teachings herein.

An outlet hose (110b) extends from cuff (110) and provides a path for fluid communication between cuff (110) and sensor (106). In the present example, sensor (106) comprises a pressure transducer such that sensor (106) is operable to sense the fluid pressure of the air in outlet hose (110b) and, hence, the pressure of cuff (110). Sensor (106) may provide an electrical signal that is representative of such fluid pressure. For instance, sensor (106) may output a voltage that varies based on the pressure encountered by sensor (106). A/D converter (101) is operable to convert an analog signal from sensor (106) into a digital signal for processing by microcontroller (105) as will be described in greater detail below. Various suitable forms that sensor (106) may take will be apparent to those of ordinary skill in the art in view of the teachings herein. Similarly, various suitable forms that A/D converter (101) may take will be apparent to those of ordinary skill in the art in view of the teachings herein.

Outlet hose (110b) further provides a path for fluid communication from cuff (110) to proportional valve (108), which is operable to release the air from cuff (110) into the atmosphere at a selectively controllable rate as described in more detail below. In the present example, proportional valve (108) is controlled by PWM driver (103). Various suitable forms that proportional valve (108) may take will be apparent to those of ordinary skill in the art in view of the teachings herein. Similarly, various suitable forms (i.e., circuit components and configurations) that PWM driver (103) may take will be apparent to those of ordinary skill in the art in view of the teachings herein.

Cuff (110) is also in fluid communication with safety valve (109) via outlet hose (110b). In the present example, safety valve (109) is configured to toggle between a fully closed state and a fully open state, where safety valve (109) is operable to rapidly release the air from cuff (110) into the atmosphere (e.g., at a faster rate than proportional valve (108)). In the present example, safety valve (109) is controlled by safety valve driver (104). Various suitable forms that safety valve (109) may take will be apparent to those of ordinary skill in the art in view of the teachings herein. Similarly, various suitable forms (i.e., circuit components and configurations) that safety valve driver (104) may take will be apparent to those of ordinary skill in the art in view of the teachings herein. In some versions, safety valve (109) is only opened when there is a power loss in system (100).

Microcontroller (105) is in communication with A/D converter (101), PWM drivers (102, 103) and safety valve driver (104). In particular, microcontroller (105) is operable to receive and process pressure data signals communicated from A/D converter (101). Microcontroller (105) is further operable to provide control signals to activate drivers (102, 103, 104), to thereby activate air pump (107) and valves (108, 109), in accordance with the control algorithm described below. Various suitable forms that microcontroller (105) may take will be apparent to those of ordinary skill in the art in view of the teachings herein.

In an exemplary operation, after cuff (110) is placed onto a limb of a subject, system (100) may be activated to cause air pump (107) to direct pressurized air into cuff (110). Sensor (106) senses the pressure inside cuff (110) and outputs an analog voltage signal, which is then digitized by A/D converter (101) and communicated to microcontroller (105). The pressure data read by microcontroller (105) may then be processed according to one or more algorithms stored in a storage device, such as a storage device present in microcontroller (105), in order to establish inflation and deflation profiles of cuff (110).

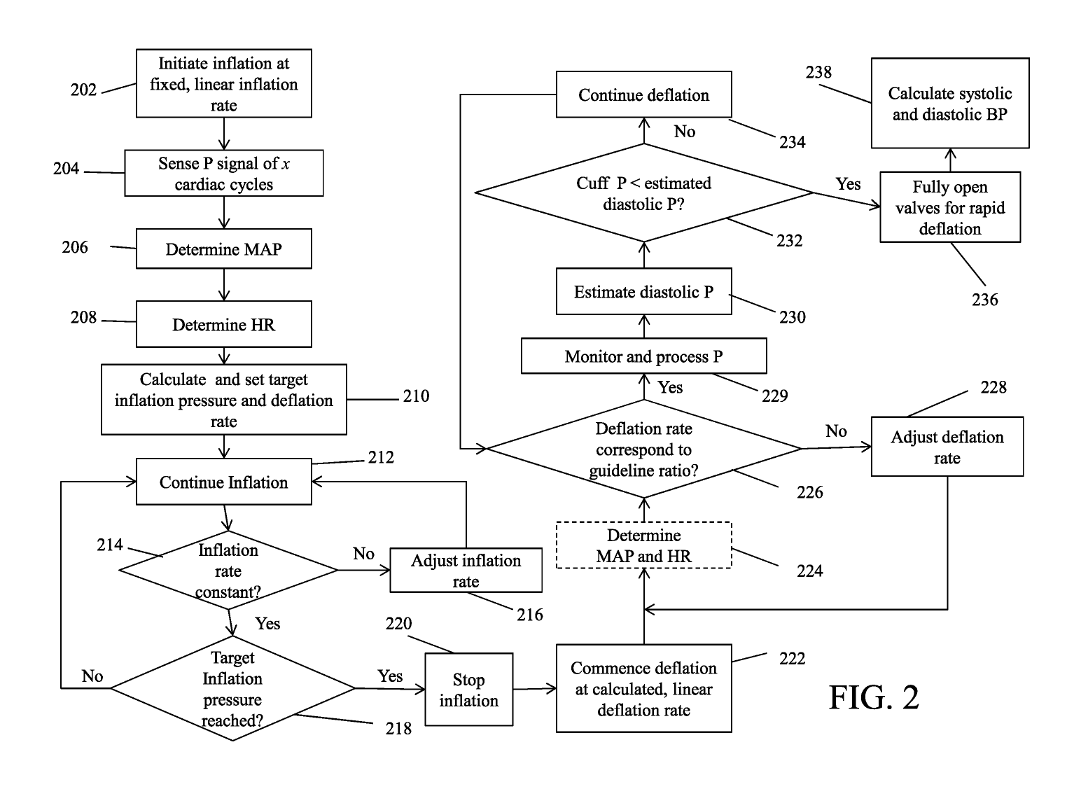

The algorithm of the software program stored in microcontroller (105), for example, controls inflation and deflation of the cuff (110) as well as the measurement and calculation of the diastolic and systolic blood pressures, discussed in detail below. In the present example, referring to FIG. 2, the algorithm is configured to complete an inflation phase in order to inflate the cuff (110) and a deflation phase in order to deflate the cuff (110). In the inflation phase, microcontroller (105) operates pump (107) via PWM driver (102) according to the algorithm to pump air into cuff (110) at a predetermined linear rate. In the deflation phase, microcontroller (105) operates proportional valve (108) via PWM driver (103) to open according to the algorithm to release air from cuff (110) at a calculated linear rate. The predetermined linear inflation rate is fixed such that the inflation rate is the same for each patient; while the linear deflation rate is variable such that the deflation rate may vary per patient because it is calculated based on data obtained from the particular patient that cuff (110) is secured to.

In the present example, the inflation phase begins by initiating inflation (block 202) by operating pump (107) to direct pressurized air into cuff (110). In some versions, microcontroller (107) inflates the cuff (110) at a higher rate than is typically used in NIBP. By way of example only, if a conventional NIBP measurement inflation rate is 10 mmHg per second, system (100) may provide an inflation rate of 15 mmHg per second. Alternatively, any other suitable inflation rate may be used. Upon inflation commencing, sensor (106) may acquire a pressure signal of the patient's cardiac cycle during a certain number (e.g., “x”) of cardiac cycles (block 204). The pressure signals are obtained for a number of cardiac cycles (x) that allows the algorithm to calculate and/or estimate the mean arterial blood pressure (MAP) and HR. Various characteristics (e.g., patient height, weight, age, etc.) may be used to determine the number of cardiac cycles (x) during which the pressure signals may be sensed by sensor (106). Alternatively, the same predetermined number of cardiac cycles (x) may be used for all patients.

In order to determine and/or estimate MAP (block 206), in the example shown, the pressure pulses of the cardiac cycles are extracted from the linear inflation pressure superimposed by the pulses. The amplitude of the pulses and the intervals between pulses are calculated. The peak of the envelope of the pulse's amplitude is identified, and the point in time where the peak occurs is also identified. The estimated MAP is the location of the maximum amplitude of the envelope of the pressure pulses. The HR is estimated using the weight-averaged interval between pulses. An interval's weight is proportional to the amplitude of the two enclosing pulses. The MAP and/or HR may be determined according to other methods as will be understood by those skilled in the art according to the teachings herein.

The estimated MAP and HR are used to determine the target pressure of inflation and the rate of deflation (block 210). In the present example, the target inflation pressure is between approximately 15 mmHg and approximately 50 mmHg higher than the estimated systolic blood pressure. Such parameters are chosen to ensure the validity of the measurement and to ensure that the duration of the measurement process is not too lengthy. The estimated systolic blood pressure (P_{systolic}) is calculated from the MAP with an empirical formula. The empirical determination of P_{systolic} is based on the principle that the difference between systolic blood pressure and MAP is positively correlated with the magnitude of MAP. One example of the empirical formula may be implemented as P_{systolic}=60+1.2*(MAP−40). Once the estimated systolic blood pressure P calculated, the inflation target pressure is set by microcontroller (105) based on MAP (block 210). The inflation target pressure is 15 mmHg or more higher than P systolic in order to acquire one or more cardiac pulses above P_{systolic}. The difference between inflation target pressure and P_{systolic} is inversely proportional to the estimated HR, not necessarily linearly. One example of an inflation target pressure determination formula may be implemented as inflation target pressure mmHg=P_{systolic}+20+5*exp((70−HR)/50). Microcontroller (105) implements a proportional-integral-derivative (PID) control algorithm to control the air pump (107) to continue inflation of the cuff (block 212) to the target pressure, which is estimated by the algorithm in real-time. Sensor (106) continuously or periodically senses the inflation pressure within cuff (110) and sends a signal to the microcontroller (105), which thereby continuously or periodically calculates the inflation rate to determine if the inflation rate remains constant (block 214). In order to keep a constant inflation rate, microcontroller (105) applies a PID algorithm, which in the present example is a negative feedback control algorithm to adjust the signal from the microcontroller (105) to PWM driver (102) to selectively control pump (107) during the inflation phase (block 216). Once the target pressure is reached (block 218), microcontroller (105) ceases operation of pump (107) to end the inflation phase, thereby stopping inflation of cuff (110) (block 220), and commences deflation (block 222). In other examples, the microcontroller (105) may be configured to maintain the inflation rate at a variable rate.

The deflation rate in the present example may be varied during deflation or may remain constant during deflation as discussed below. In either case, the deflation rate is ad hoc per patient. The ad hoc deflation rate may be at least initially selected based on a calculation according to different characteristics of the patient, such as, for example, HR and/or MAP. Other characteristics, such as height, weight, age, etc., may be used to determine the ad hoc deflation rate. In the present example, the HR is used to calculate the ad hoc deflation rate with the goal of maintaining a certain predetermined ratio of cuff pressure decrease per each pulse cycle. Therefore, the HR as determined in block (208) may be used in this determination or, optionally, the HR (and MAP) may be determined again at block (224).

In the present example, the deflation rate is determined and controlled in order to obtain a guideline ratio of at least one pulse for each 10 mmHg of difference in deflation pressure between the systolic and diastolic blood pressure. If the HR can be reasonably estimated when the deflation starts (e.g., at block 224), the deflation rate can be determined by the formula: deflation rate (mmHg/second)<HR (beats/second)/6. By way of example, under the above one pulse/10 mmHg guideline, the deflation rate should be 6.7 mmHg per second or less if the heart is 40 beats per second. Similarly, the deflation rate would be 20 mmHg per second or less if the HR is 120 beats per second under the above guideline. In other examples, as the guideline ratio deviates from the 10 mmHg/pulse, the deflation rate should deviate accordingly.

Similar to the inflation phase, the algorithm of microcontroller (105) implements a proportional-integral-derivative (PID) control scheme during the deflation phase. Sensor (106) continuously or periodically senses the deflation pressure within cuff (110) and sends a signal to the microcontroller (105), which thereby continuously or periodically calculates the deflation rate to determine if the deflation rate remains within the guideline ratio discussed above (block 226). In order to keep the deflation rate at a deflation rate to remain within the guideline ratio, microcontroller (105) applies the PID algorithm/control scheme, which in the present example is a negative feedback control algorithm to adjust the signal from the microcontroller (105) to the proportional valve (108) via PWM driver (103), and to thereby adjust the deflation rate (block 228) of cuff (110). In an instance where the deflation rate does remain within the guideline ratio as described herein, the deflation rate need not be adjusted, the pressure is monitored and processed (block 229) in order to estimate diastolic pressure (230), discussed in more detail below. Although in some instances the deflation rate will be variable such that it will need to be adjusted from time to time in order to remain within the guideline ratio, it is possible that the deflation rate will remain within the guideline ratio during the entire deflation phase, thus making the deflation rate effectively constant during the deflation phase.

When the pressure in the cuff (110) reaches a level of pressure adequately lower than the estimated diastolic blood pressure (P_{diastolic}), the deflation process may end and the cuff (110) may be instantly discharged by fully opening the valves (108 and/or 109) to end the measurement duration. Estimated diastolic pressure may be calculated (block 230) per the equation, P_{diastolic}=(3*MAP−P_{systolic})/2 and the aforementioned formula P_{systolic}=60+1.2*(MAP−40). Once the pressure in the cuff (110) reaches a level of pressure adequately lower than the estimated diastolic blood pressure (block 232), as calculated in the present example as described above, the rate-controlled deflation process may end (block 236). The deflation end pressure is 15 mmHg or more lower than P_{diastolic}, in order to acquire one or more cardiac pulses below P_{diastolic}. The difference between the P_{diastolic} and the deflation end pressure is inversely proportional to HR, not necessarily linearly. One embodiment of the deflation end pressure may be implemented as deflation end pressure mmHg=P_{diastolic}−(20 5*exp((70−HR)/50)). At the deflation end pressure the controller (105) may fully open one or both of the valves (108, 109) and rapidly deflate the air from cuff (110), in order to end the measurement duration.

After the blood pressure signal is obtained, the systolic and diastolic blood pressures may be calculated (block 238). In the present example, the algorithm described herein that is operative to control the inflation and deflation of cuff (110) may also use a series of signal processing methods and oscillometric method of NIBP measurement equations to compute the systolic and diastolic blood pressures. In other examples, however, other algorithms stored by microcontroller (105), or another controller or storage device may implement the calculation of the systolic and diastolic blood pressures.

In the example shown, the pressure signal is low-pass filtered and high frequency noise is removed from the signal. The noise-free signal is baseline corrected by a polynomialfitting algorithm. Pulses of cardiac cycles are extracted from the baseline corrected signal by the algorithm or other algorithms which is/are able to locate the peaks and valleys of the pulses. Motion artifacts are identified with multiple criteria and excluded from the true cardiac pulses. The artifact criteria include amplitude, slope, shape and duration. The amplitudes and time location of the pulses are used to construct an envelope of the pulses waveforms. The peak amplitude of the envelope is then located and determined. A pair of percentage values (systolic threshold and diastolic threshold) are used for the specific MAP to derive the systolic and diastolic blood pressure. Multiple pairs of thresholds are preset for multiple ranges of MAP. The peak amplitude of the cardiac pulses is used as a reference point. The systolic pressure is determined when the cardiac pulse amplitude is at the systolic threshold percentage of the envelope peak above the MAP. The diastolic pressure is determined when cardiac pulse amplitude is at the diastolic threshold percentage of the envelope peak. Once calculated, the systolic and diastolic blood pressures may be displayed on a display device.

Thus, when a subject's HR is low, the deflation rate is adjusted down to a lower rate to record as many cardiac cycles as when the HR is normal (i.e., the predetermined number of cardiac cycles (x)). When the HR is high, the deflation rate is adjusted to a higher rate so that the measurement process stops as soon as the predetermined number of cardiac cycles (x) have occurred, thereby minimizing the duration of the measurement process. Minimizing the duration of the measurement process may be particularly useful in pediatric patient populations, since pediatric patients may tend to have a higher HR and it may be more difficult to restrain their body movements that might otherwise cause severe artifacts in NIBP signal and lower the measurement accuracy.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/we claim:

1. A method for measuring blood pressure with a non-invasive blood pressure monitor, the method comprising:
(a) measuring a patient heart rate;
(b) determining a blood pressure cuff deflation rate based on at least the measured patient heart rate;
(c) inflating a blood pressure cuff while the blood pressure cuff is worn by a patient;
(d) deflating the blood pressure cuff at the determined blood pressure cuff deflation rate;
(e) monitoring a pressure of the blood pressure cuff during the act of inflating and during the act of deflating; and
(f) determining the patient's blood pressure based on the pressure of the blood pressure cuff as monitored during the act of inflating and during the act of deflating.

2. The method of claim 1, wherein the blood pressure cuff is inflated to a target cuff pressure, the method further comprising determining the target cuff pressure based on at least the measured patient heart rate.

3. The method of claim 1, wherein the current cuff pressure is monitored for a number of cardiac cycles, wherein the number of cardiac cycles is determined based upon:
(i) a patient height,
(ii) a patient weight, and
(iii) a patient age.

4. The method of claim 1, wherein the blood pressure cuff is inflated to a target cuff pressure between about 15 mmHg and about 50 mmHg greater than a systolic blood pressure estimate.

5. The method of claim 1, wherein the non-invasive blood pressure monitor comprises a microcontroller, an air pump, and a proportional valve, wherein the act of inflating the blood pressure cuff is performed using the air pump, wherein the act of deflating the blood pressure cuff is performed using the proportional valve, and wherein the microcontroller operates the air pump and the proportional valve.

6. The method of claim 1, wherein the blood pressure cuff deflation rate allows for at least one cardiac pulse at each stage of a set of deflation stages, the method further comprising:
(a) determining a diastolic blood pressure estimate; and
(b) when the set of pressure data indicates that the current cuff pressure of the blood pressure cuff is less than the diastolic blood pressure estimate, deflating the blood pressure cuff at a rapid deflation rate.

7. The method of claim 6, further comprising the step of adjusting the blood pressure cuff deflation rate when the blood pressure cuff deflation rate does not allow at least one cardiac pulse at each stage of the set of deflation stages.

8. The method of claim 1, wherein the act of determining the blood pressure cuff deflation rate comprises:
(i) dividing the patient heart rate by six to produce a deflation guideline, and
(ii) setting the target deflation rate to a value less than or equal to the deflation guideline in mmHg per second.

9. A method for measuring blood pressure with a non-invasive blood pressure monitor, the method comprising:
(a) configuring a microcontroller to operate an air pump and an air release, wherein the air pump is operable to inflate a blood pressure cuff, wherein the air release is operable to deflate the blood pressure cuff;
(b) configuring the microcontroller to receive a set of pressure data from a pressure sensor and determine a set of blood pressure data from the set of pressure data, the set of blood pressure data comprising a mean arterial pressure, a systolic blood pressure estimate, and a patient heart rate;
(c) executing at the microcontroller a set of inflation instructions causing the air pump to inflate the blood pressure cuff to a target inflation pressure, wherein the target inflation pressure is based upon the systolic blood pressure estimate;
(d) executing at the microcontroller a set of deflation instructions causing the air release to deflate the blood pressure cuff at a target deflation rate, wherein the target deflation rate is based upon the patient heart rate; and
(e) executing at the microcontroller a set of blood pressure calculation instructions producing a systolic blood pressure measurement and a diastolic blood pressure measurement.

10. The method of claim 9, wherein the act of determining the set of blood pressure data comprises:
(i) determining a number of cardiac cycles for which to gather the set of pressure data,
(ii) determining the mean arterial pressure by:
(A) extracting a set of pressure pulses from the set of blood pressure data,
(B) identifying a set of amplitudes of the pressure pulses,
(C) identifying a set of intervals of the set of pressure pulses,
(D) identifying an envelope of the set of amplitudes, and
(E) identifying the location of a peak amplitude of the envelope,
(iii) determining the systolic blood pressure estimate based upon the mean arterial pressure, and
(iv) determining the patient heart rate based upon the set of intervals.

11. The method of claim 10, wherein the number of cardiac cycles is determined based upon:
(A) a patient height,
(B) a patient weight, and
(C) a patient age.

12. The method of claim 9, wherein executing the set of inflation instructions further causes:
(i) the air pump to inflate the blood pressure cuff at an initial linear rate,
(ii) a determination of whether the initial linear rate is resulting in a constant rate of inflation of the blood pressure cuff,
(iii) when the blood pressure cuff is not inflated at a constant rate of inflation, an adjustment of the initial linear rate to achieve a constant rate of inflation, and
(iv) when the target inflation pressure is reached, a cessation of the air pump.

13. The method of claim 12, wherein the target inflation pressure is determined as being between about 15 mmHg and about 50 mmHg greater than the systolic blood pressure estimate.

14. The method of claim 9, wherein executing the set of deflation instructions further causes:
(i) the blood pressure cuff to deflate at the target deflation rate, wherein the target deflation rate allows for at least one cardiac pulse at each stage of a set of deflation stages,
(ii) when the target deflation rate does not allow at least one cardiac pulse at each stage of the set of deflation stages, an adjustment of the target deflation rate,
(iii) determination of a diastolic blood pressure estimate, and
(iv) when the set of pressure data indicates that a pressure of the blood pressure cuff is less than the diastolic blood pressure estimate, a rapid deflation of the blood pressure cuff.

15. The method of claim 14, wherein the act of determining the target deflation rate comprises:
(i) dividing the patient heart rate by six to produce a deflation guideline, and
(ii) setting the target deflation rate to a value less than or equal to the deflation guideline in mmHg per second.

16. The method of claim 14, wherein the systolic blood pressure estimate is determined by the equation 60+(1.2*(the mean arterial pressure−40)).

17. The method of claim 16, wherein the diastolic blood pressure estimate is determined by the equation ((3*the mean arterial pressure)−the systolic blood pressure estimate)/2.

18. A non-invasive blood pressure monitor comprising:
(a) a blood pressure cuff adapted to fit a patient;
(b) a pump operable to inflate the blood pressure cuff;
(c) a valve operable to deflate the blood pressure cuff;
(d) a pressure sensor configured to generate a set of pressure data; and
(e) a microcontroller configured to operate the pump and the valve, wherein the microcontroller is further configured to determine a set of blood pressure data based upon the set of pressure data, the set of blood pressure data comprising a mean arterial pressure, a systolic blood pressure estimate, and a patient heart rate;
wherein the microcontroller is configured to execute:
(i) a set of inflation instructions, wherein executing the set of inflation instructions causes the pump to inflate the blood pressure cuff to a target inflation pressure, wherein the target inflation pressure is based upon the systolic blood pressure estimate,
(ii) a set of deflation instructions, wherein executing the set of deflation instructions causes the valve to deflate the blood pressure cuff at a target deflation rate, wherein the target deflation rate is based upon the patient heart rate; and
(iii) a set of blood pressure calculation instructions, wherein executing the set of blood pressure calculation instructions produces a systolic blood pressure measurement and a diastolic blood pressure measurement.

19. The non-invasive blood pressure monitor of claim 18, wherein the microcontroller is configured to:
(i) determine a number of cardiac cycles for which to gather the set of pressure data,
(ii) determine the mean arterial pressure by executing instructions to:
(A) extract a set of pressure pulses from the set of blood pressure data,
(B) identify a set of amplitudes of the set of pressure pulses,
(C) identify a set of intervals of the set of pressure pulses,
(D) identify an envelope of the set of amplitudes, and
(E) identify the location of a peak amplitude of the envelope,
(iii) determine the systolic blood pressure estimate based upon the mean arterial pressure, and
(iv) determine the patient heart rate based upon the set of intervals.

20. The method of claim 19, wherein the microcontroller is configured to determine the number of cardiac cycles based upon:
(i) a patient height,
(ii) a patient weight, and
(iii) a patient age.
